(19) United States
(12) Patent Application Publication
(10) Pub. No.: US 2005/0045190 A1
(43) Pub. Date: Mar. 3, 2005

(54) Method and system of treating sleep disorders

(76) Inventor: Janet Bennett, Candler, NC (US)

Correspondence Address: BURNS DOANE SWECKER & MATHIS L L P, POST OFFICE BOX 1404, ALEXANDRIA, VA, 22313-1404, US

(21) Appl. No.: 10/651,546

(22) Filed: Aug. 29, 2003

(51) Int. Cl. A61B 19/00

(52) U.S. Cl. 128/898

(57) ABSTRACT

Non-surgical methods for treating sleep disorders, such as snoring or sleep apnea symptoms. The method includes selecting a patient having a sleep disorder, and instructing the patient to perform tongue exercises directed to positioning the tongue in a predetermined position against the hard palate behind the upper central two front teeth.

### BACKGROUND OF THE INVENTION

1. Field of the Invention

This invention relates to a method and system of treating sleep disorders including sleep apnea and snoring, and more particularly a non-surgical method for treating sleep disorders such as sleep apnea or snoring through a series of tongue exercises to change the resting position of the tongue while sleeping.

2. Description of the Related Art

Sleep apnea is a sleep-related breathing disorder that is thought to affect between 1-10% of the adult population. Sleep apnea is typically characterized by daytime hypersomnolence, morning headaches, choking, gasping and holding breath during sleep, intellectual deterioration, cardiac arrhythmias, snoring and thrashing during sleeping. It is caused by frequent episodes of apnea during the patient's sleep. The syndrome is classically divided into two types. One type, termed “central sleep apnea syndrome”, is characterized by repeated loss of respiratory effort. The second type, termed “obstructive sleep apnea syndrome”, is characterized by repeated apneic episodes during sleep resulting from obstruction of the patient's upper airway or that portion of the patient's respiratory tract, which is cephalad to, and does not include the larynx.

Treatment for sleep apnea traditionally includes various positional, behavioral, non-surgical or surgical therapies. For example, positional therapies include finding ways to stay on your side while you sleep, which hopefully prevents the tongue from blocking your airway. This can be done by wearing a backpack with a towel in it or by sewing a tennis ball in a pocket on the back of your pajama top. In addition, other positional therapies include wearing a neck collar that keeps the airway open by keeping the chin up and the head tilted back, wearing a devise that supports your jaw, or raising the head of the bed about four inches.

Meanwhile, behavioral therapy includes losing weight for the purpose of getting rid of excess, flabby tissue in the throat. In addition, behavioral therapy can include reducing alcohol/tranquilizer consumption, since alcohol can cause muscle relaxation that can reduce the force of the muscles that help to keep your airway open, and stop smoking which can irritate the nasal airway. Although, behavioral therapy is a safe alternative to surgical procedures and other therapies, it is often very difficult for the patient to make such lifestyle changes and the patient rarely achieves such changes.

The traditional non-surgical therapies are typically devices that bring the jaw forward, and elevate the soft palate or keep the tongue from falling back in the airway. The non-surgical devices include oral/dental appliances that displace the tongue forward or move the mandible to an anterior and forward position to open the airway. However, patients often report that devices can cause side effects include excessive salivation and temporomandibular joint discomfort. Other devices include the use of a nasal support devise that prevents the collapse of the sides of the nose during sleep, a mandibular advancement device which clasps onto the upper and lower teeth and pulls the jaw forward. One of the problems with mandibular devices is that they often require adjustment, which can take a long time and require a lot of trial and error.

Another set of devices includes the use of adhesive strips to hold your nostrils open, and Continuous Positive Airway Pressure (CPAP). The CPAP device pumps air into the upper airway through a mask worn over the nose (in some cases, nose and mouth) during sleep. The mask is connected to a blower forcing air through the nasal passages. It increases the pressure and opens the upper airway. Disadvantages to using the CPAP include irritation to the throat and nose, sinus problems, masks that don't fit properly, noise of the machine and it is costly.

Surgical procedures include Uvulopalatopharyngoplasty (UPPP), which reduces or reshapes the uvula and posterior soft palate in order to enlarge the oropharyngeal airway. The Laser-Assisted Uvulopalatoplasty (LAUP) is similar to the UPPP, but it is an outpatient surgical procedure involving several sessions spaced over several weeks time. Less tissue is removed than by the UPPP surgery. Somnoplasty is a process that “cooks”, or shrinks, unwanted fat in the throat with radio-frequency energy. Repose, known as tongue suspension procedure: it is intended to keep the tongue from falling back over the airway during sleep with a small screw inserted into the lower jaw bone and stitches below the tongue.

Meanwhile, nasal surgery is often performed to eliminate nasal obstructions including Tonsillectomy and/or Adenoidectomy or Partial Glossectomy and Linguoplasty. Nasal surgery is often necessary to remove enlargements in the airway at the back of the tongue. One of the last choices is a permanent tracheostomy which is a surgical opening made into the trachea for airway management. Most of the surgical procedures may be effective, but are often risky and costly which is unacceptable to a patient.

While the treatment and therapies described in the prior art are numerous, none of the therapies were designed to train the tongue muscles to reposition the tongue naturally. Accordingly, what was needed is a non-surgical method and system of naturally treating sleep apnea and other related symptoms by training the tongue muscles to reposition the tongue so that the tongue does not obstruct the patient's airway.

### SUMMARY OF THE INVENTION

The present invention relates to a non-surgical method for treating sleep disorders such as sleep apnea and snoring by instructing a sufferer to perform a series of exercises to train the tongue to rest on the roof of the mouth immediately in back of the medial incisors.

In accordance with one aspect of the invention, presented is a non-surgical method for treating sleep disorders such as snoring or sleep apnea which includes instructing patient having a sleep disorder to perform tongue exercises directed to positioning the tongue in a predetermined location on the hard palate behind the upper front teeth.

In accordance with another aspect of the invention, presented is a non-surgical method for treating sleep disorders such as snoring or sleep apnea symptoms which includes instructing a patient having a sleep disorder to perform tongue exercises directed to positioning the tongue in a predetermined location on the hard palate and directly behind the upper front teeth; and instructing the patient to perform at least one additional tongue exercise selected from the group consisting of:
a. an exercise for strengthening the muscles of the tongue;
b. an exercise for stimulating the muscles of the tongue;
c. an exercise for controlling the muscles of the tongue;
d. an exercise for teaching the muscles of the tongue to rest in the predetermined location during everyday activities.

In accordance with a further aspect of the invention, presented is a non-surgical method of treating sleep disorders such as snoring or sleep apnea which includes instructing a patient having a sleep disorder to perform tongue exercises that train the patient's tongue to rest against the hard palate directly behind the upper front teeth, wherein the patient performs at least one of the following exercises at least once a day for at least two days:
a. tongue up on the roof;
b. tongue tip exercises;
c. tongue strokes.

In accordance with another aspect of the invention, presented is a method for treating sleep disorders by training a person's tongue to rest against a roof of the person's mouth, the method including the person performing at least one of the following exercises at least once a day for at least two days:
an exercise for teaching the tongue to rest against the roof of the mouth;
an exercise for strengthening the muscles of the tongue;
an exercise for stimulating the muscles of the tongue;
an exercise for controlling the muscles of the tongue; and
an exercise for training the tip of the tongue to rest against the roof of the mouth during everyday activities.

In accordance with a further aspect of the invention, presented is a non-surgical method for treating sleep disorders such as snoring or sleep apnea which includes performing a first series of tongue exercises to train a patient's tongue to rest on the hard palate directly behind the upper front teeth; and repeating the first series of tongue exercises daily for at least two days.

In accordance with another aspect of the invention, presented is a non-surgical method for treating sleep disorders such as snoring or sleep apnea, the method including the steps of: performing at least one exercise for training the tongue to rest against a roof of a mouth, wherein the at least one exercise is performed at least once a day, wherein at least one exercise includes one of the following: Tongue up on the Roof, Tongue Tip Exercise, Tongue Strokes, Slurp and Swallow up on the Roof, Tongue Taps, Tense and Relax, Activities up on the Roof×3 (or ×4), Gargle, Slow Tongue Pops, Sleep Right, Rest Posture, Trapped up on the Roof, Tongue Drags on the Roof, Stretches up on the Roof, Where is your Tongue?, Tongue Kicks, and Back Tongue Pressure.

In accordance with a further aspect of the invention, presented is a non-surgical method for treating sleep disorders which includes instructing a patient having a sleep disorder to perform an exercise directed to positioning the tongue in a predetermined location on the hard palate behind the upper front teeth by performing an exercise which includes placing the tip of the patient's tongue against the patient's hard palate right behind the upper front teeth, and holding the tip of the tongue in that position for at least about 30 seconds.

### BRIEF DESCRIPTION OF THE DRAWINGS

The invention will now be described in greater detail with reference to the various embodiments illustrated in the accompanying drawings, in which like elements bear like reference numerals, and wherein:

FIG. 1 is a side view of a patient's mouth with the tip of the tongue resting against the hard palate, just behind the upper front teeth.

### DETAILED DESCRIPTION OF THE INVENTION

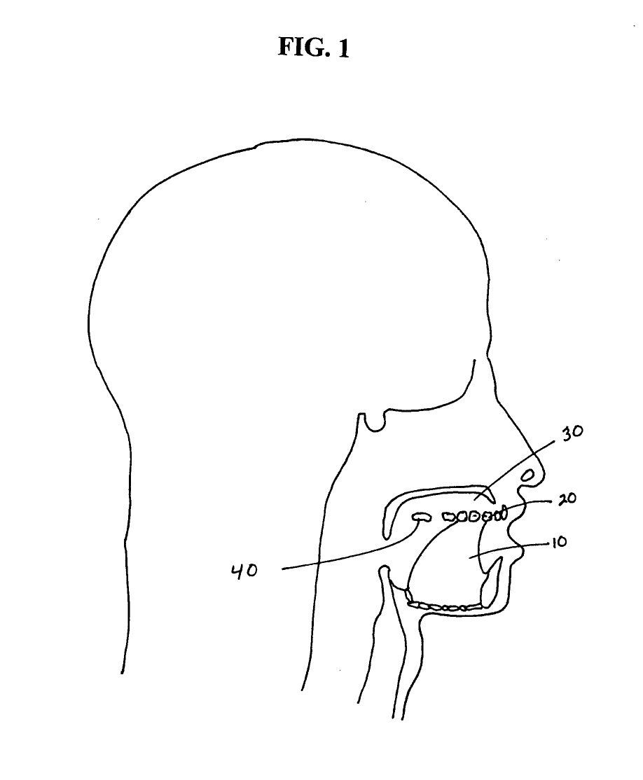

The present invention relates to a non-surgical method and system of naturally treating sleep disorders such as snoring and sleep apnea symptoms by training the tongue muscles to reposition the tongue so that the tongue does not obstruct the patient's airway. As shown in FIG. 1, the method and system trains the tongue 10 to rest the tip 20 of the tongue on the roof of the mouth 30, just behind the teeth 40, when one is not eating, drinking or talking. This contact point is referred to as the “spot.” The spot is the same place that one would place his or her tongue if making the T or D sound. For example, when saying the word “door” out loud, the tongue should push against the roof of the mouth, right behind the teeth. By training the tip 20 of the tongue 10 to rest against the roof of the mouth 30, snoring and sleep apnea related symptoms can be treated without devices or surgical means.

The method and system comprises the steps of performing a series of exercises for teaching the tongue to rest against a roof of a mouth; strengthening the tip of the tongue, the blade (middle) of the tongue, and the back of the tongue; strengthening the muscles that pull back the tongue; strengthening the muscles that narrow the tongue; stimulating the muscles in the blade of the tongue; controlling the muscles in the side of the tongue; teaching the muscles to relax in the back of the tongue; teaching the tip of the tongue to relax against the roof of the mouth during everyday activities; teaching the tip of the tongue to relax against the roof of the mouth while sleeping; practicing the correct position of the tongue, lips and mandible when an individual is not eating or talking; and stimulating the muscles in the back of the tongue.

Each exercise is typically performed with at least one or two other exercises, wherein each of the exercises complements the other exercises performed with each lesson. For example, one exercise may emphasize the location of the tip of the tongue, while the other exercise emphasizes a strengthening aspect of the tongue.

The exercises are preferably practiced at least twice a day for at least one week in a defined lesson plan. Each lesson of several exercises teaches or trains the tongue to repeat the positioning of the tongue to avoid snoring and sleep apnea symptoms. However, it can be appreciated that the exercises can be performed at any regularity without departing from the present invention. Each lesson prepares you for the next lesson. If, for some reason, you do not do the exercises two times daily for a particular lesson, it is preferable that one does not proceed to the next lesson until you have completed the previous one. Each lesson is preferably performed for at about 3 to 10 days and more preferably at least 7 days, however, it can be appreciated that each lesson can be performed at other frequencies without departing from the present invention.

The patient can receive instruction on the systems and methods for treating sleep disorders in a face-to-face setting, or by written material, video or DVD, or via the Internet.

A list of the exercises is recited including the purpose of each exercise and how to perform the exercise including correct positioning of the tongue are set forth. It can be appreciated, that minor changes to the exercises can be performed without departing from the present invention.

List of Exercises:

Tongue up on the Roof

The tongue up on the roof exercise teaches the patient to place his or her tongue in the correct position resting the tip of the tongue against the hard palate, just behind the teeth. The exercise begins by placing the tip of the tongue tip on the spot, back teeth together, or slightly apart, and lips closed. When the patient closes their lips, the front ¼ to ⅓ of the tongue should be touching the roof of the mouth. When performing this exercise, it is preferable that the back part of the tongue does not make contact with the roof of the mouth. In addition, the tongue should rest comfortably. Preferably, the patient will hold it there for at least 30 seconds to 5 minutes each time they practice, and more preferably at least 60 seconds. As the saliva builds, the patient should swallow as necessary. However, it is preferable that the tongue tip does not slip off the spot when swallowing.

Tongue Tip Exercises

This exercise will strengthen the tip of the tongue. The patient begins the exercise by placing the tip of his or her tongue on the spot and sucks in slightly and repeatedly. The patient's lips should be apart, and the teeth are NOT together. One should not pop the tongue. Instead it is preferable that only the tip of the tongue is used for this exercise, as if making a scolding sound. The exercise preferably is performed in groups of at least 50 clicks—as fast as possible—each time the patient practices. However, it can be appreciated that one does not really need to count them as long as one just does a lot of them! It should take less than one minute.

Tongue Strokes

This exercise will accomplish two things. It will strengthen the muscles that pull the tongue back and the muscles that narrow the tongue. First of all, find an object that is elongated and lightweight, such as a straw, a swizzle stick, a chopstick, or the end of a toothbrush, etc. The patient sticks his or her tongue out and lets it relax on the patient's bottom lip. The patient then places the straw (or whatever the patient is using) on the side (edge) of the tongue all the way in the back. Lightly stroke it all the way down to the tip of the tongue, stopping at the tip. Do not go from the tip to the back, only from the back to the tip. This may tickle! The tongue may also be jumpy! Alternatively, the tongue may do nothing at all. The use of a mirror may be preferred to insure that the object is on the side of the tongue and not on the top or bottom. In one embodiment, it is preferable that this exercise is performed when brushing ones teeth on an at least twice a day basis. Preferably, the patient strokes both sides at least 10 times each session.

Slurp and Swallow up on the Roof

The purpose of this exercise is to train the tongue to feel comfortable making contact on the roof of the mouth and still maintain a good swallow position. Bite the back teeth together, or keep them slightly apart, and smile real big, so the teeth show. Put the tongue tip on the spot. Now slurp the saliva and swallow. Do not let the tongue tip move from the spot. The slurping provides some moisture to swallow and helps to suction the tongue up on the roof of the mouth. In one embodiment, it may be preferable to get a glass of water and take sips in between slurps, as the mouth will get very dry. Do not slurp with water in the mouth. Hint: If the patient is having difficulty with this exercise, he or she may be trying too hard. The slurp does not have to be a loud and long slurp. As soon as you slurp, the tongue is suctioned up on the roof of the mouth and the patient should swallow immediately. Do this exercise two times daily. Do it 5 times at each practice session.

Tongue Taps

The purpose of this exercise is to stimulate the muscles in the blade of the tongue. Using the swizzle stick, chopstick, straw, spoon, or whatever item the patient chose to use in the last lesson, stick out the tongue and tap firmly on the top of the blade (middle section) of the tongue sixty (60) times. Tap on the middle horizontal portion of the tongue—not the tip or the back—the middle portion. Practice it two times daily—that means 120 times daily.

Tense and Relax

This exercise will help the patient learn to control the muscles in the sides of the tongue. USE A MIRROR. Stick out the tongue and let it rest on the bottom lip. Tense the muscles so that the tongue is very narrow and pointed. The sides of the tongue are not curled up. The patient should pretend that they are licking the chocolate icing off of the beater! Hold it there for 3 seconds. Now relax the muscles and hold it there for 3 seconds. Tense the tongue again for 3 seconds, then relax the tongue for 3 seconds. Go back and forth. Do this 10 times at each practice session—two times daily. Activities up on the Roof×3 (or ×4)

The purpose of this exercise is to help the patient get used to holding the tongue tip on the spot while the patient performs simple activities during the day. Each day, keep the tongue tip on the spot while doing 3 activities. These are activities that require concentration, making it more challenging for the patient to know where the tongue tip is resting. The patient will probably have to consciously put the tongue tip on the spot. The teeth should be together and the lips should be closed. The entire tongue should be flattened up against the roof of the mouth.

This exercise is very important because the patient will purposefully plan to keep the tongue tip on the spot for 5 minutes—3 times per day (4 times per day). This will make the patient begin to be more aware of where the tongue tip rests when you are not talking or eating. Examples of activities: taking a shower, shaving, getting dressed, making the bed, washing dishes, cooking, any type of cleaning activity, working on the computer, etc. Driving, watching TV, and reading do not qualify for this exercise. The patient preferably will be performing an activity, not just sitting. Of course, the exercise may be done while just sitting.

Gargle

Gargling is a good exercise to help the patient relax and strengthen the muscles in the back of the tongue. The patient preferably will gargle every time he or she brushes his or her teeth. The patient can gargle with mouthwash, plain water or with nothing in the mouth.

Slow Tongue Pops

This exercise is very important in helping to strengthen the muscles in the blade (middle) of the tongue. Suction the entire tongue up on the roof of the mouth, like when you popped your tongue as a child. Look in the mirror and be sure that the lingual frenum (the muscle under the tongue) is stretched. The mouth must be open but not real wide. Hold it there for 5 seconds and then pop it down slowly and forcefully. It doesn't have to be loud. Use the entire tongue, not just the tongue tip. Pop the tongue 20 times each time the patient practices. Practice two times daily.

Sleep Right

The purpose of this exercise is to train the tongue to stay on the spot while the patient sleeps. This is very important. The patient will accomplish this by training the subconscious mind to keep the tongue in the correct position while sleeping. When the patient is ready to go to sleep (after the patient has watched TV or listened to the radio or read a book), put the tongue tip on the spot and try to get the entire tongue flattened up against the roof of the mouth as much as possible while still feeling comfortable. Try to create a vacuum between the tongue and the roof of the mouth. The patient should tell himself “tongue on the spot, tongue on the spot, tongue on the spot”. Close the eyes and go to sleep! This exercise is preferably performed every night! This is the way the subconscious mind learns—it needs repetition, repetition, and repetition.

Rest Posture

Where the tongue “lives” in the mouth when the patient is not talking or eating is very important. Accordingly, the purpose of this exercise is to practice the correct resting posture of the tongue, lips and mandible. Raise the tongue tip to the spot. Close the teeth and lips. The patient may keep the teeth slightly apart if it is uncomfortable to keep them closed. Never clench the teeth. The entire tongue should be flattened up against the roof of the mouth (or as much as possible to still remain comfortable). Maintain this correct resting posture while the patient watch one 30-minute TV program.

Trapped up on the Roof

This exercise will help the tongue become more used to staying flattened up against the roof of the mouth. Take a small sip of water. Put the tongue tip on the spot and raise the entire tongue against the roof of the mouth. Open the mouth and lean the head down to be sure that the tongue has formed a tight seal around the gum ridge. If no water drips out, the patient has done it correctly. Now, raise the head, bite the teeth together and swallow. Be sure the tongue tip does not slip off the spot when the patient swallows. This exercise is preferably performed about 10 times each time, the patient practice. Practice two times daily. Hint: If the patient has difficulty with this exercise, the patient has probably taken a large sip. Try again with a smaller sip of water.

Tongue Drags on the Roof

Drag the tongue tip back along the hard and soft palate. Go as far as the patient can. Try it with the mouth open initially and then do it with the lips together. Do 10 tongue drags at each practice session for a total of 20 per day.

Stretches up on the Roof

This exercise is similar to the slow tongue pops in Lesson 4. Suction the entire tongue up on the roof of the mouth and be sure that the lingual frenum (the muscle under the tongue) is stretched. Do NOT pop the tongue. While the tongue is up, open and close the jaw. Do this as many times as the patient can before the tongue falls down. Start with 10 stretches and increase the number until the patient can easily do 100 without stopping. Do this exercise two times daily.

Where is the Tongue?

The tongue should be on the spot and the teeth and lips should be closed at all times when the patient are not talking or eating. The tongue should be flattened against the roof of the mouth as much as possible to remain comfortable. Start thinking about this!

Tongue “Kicks”

The purpose of this exercise is to strengthen the back muscles of the tongue. When the patient says “kick”, the back of the tongue is humped up against the soft palate. Look in the mirror and watch the back of the tongue as the patient says, “Kick”. Raise and lower the back of the tongue 20 times at each practice session by saying “kick”, producing a hard “k” sound. Do this exercise two times daily.

Back Tongue Pressure

The purpose of this exercise is to stimulate the back of the tongue and make it easier to go up to the roof of the mouth. Place the fingertip on the back of the tongue, in the middle. If the patient goes back too far, the patient may gag. Apply constant pressure with the index finger. The tongue should resist this pressure. Hold for 5 seconds. Release. Do this exercise 20 times each time the patient practice. Be sure to practice 2 times daily.

Sample Exercise Plan:

Each of the lessons provides a specific exercise for the tongue to be trained to rest up against the roof of the mouth even when the patient is not thinking about it. The exercises are performed in lessons of at least two exercises. One example of a method of treating snoring and sleep apnea symptoms is as follows:

### Lesson 1

The purpose of lesson 1 is that when you are not eating, drinking or talking, your tongue tip should rest against the hard palate, just behind your teeth. The contact point or spot 30, is the same place you should make the T or D sound. Say the word “door” out loud. Where does your tongue push when you make the “D” sound? Your tongue should not push against your teeth. It should push on the roof of your mouth, right behind your teeth. This is the spot 30. It's important that you practice these exercises daily and more preferably at least TWO times per day. Each lesson should be practiced for a minimum of about one week; however, the length of each lesson can vary depending on each individual patient. In addition, it is preferable, if the patient is unable to practice adequately, that the patient does not advance to the next lesson until they have performed the exercises for about seven days. However, it can be appreciated that the frequency of the exercises and the duration of the lessons will vary from individual to individual.

### Exercise 1

Tongue up on the Roof

Put your tongue tip on the spot, back teeth together, or slightly apart, and lips closed. When you close your lips, the front ¼ to ⅓ of your tongue should be touching the roof of your mouth. The back part of your tongue may not make contact with the roof of your mouth. Let it rest comfortably. Hold it there for 5 minutes each time you practice. As your saliva builds, you will need to swallow. Be sure that your tongue tip does not slip off the spot when you swallow.

### Exercise 2

Tongue Tip Exercises

This exercise will strengthen the tip of your tongue. Put your tongue tip on the spot and suck in slightly and repeatedly. Your lips are apart and your teeth are NOT together. Do not pop your tongue. Use only the tip of the tongue for this exercise, as if you are making a scolding sound. Do 50 clicks—as fast as you can—each time you practice. Don't really count them—just do a lot of them! It should take less than one minute.

### Exercise 3

Tongue Strokes

This exercise will accomplish two things. It will strengthen the muscles that pull your tongue back and the muscles that narrow your tongue. Find an object that is lightweight and long, such as a straw, a swizzle stick, a chopstick, an eyebrow pencil, the arm of your glasses, a closed pen, the end of your toothbrush, etc. Stick your tongue out and let it relax on your bottom lip. Put the straw (or whatever you are using) on the side (edge) of your tongue all the way in the back. Don't put it so far back that you anticipate gagging. Be sure it is on the side of your tongue. Lightly stroke it all the way down to the tip of your tongue, stopping at the tip. Do not go from the tip to the back, only from the back to the tip. This may tickle! Your tongue may be jumpy! Your tongue may do nothing at all. USE A MIRROR because it might be difficult to feel that you are really on the side of your tongue and not on the top or bottom. It might be best to do this when you brush your teeth twice daily. Stroke both sides 10 times each time you practice.

### Lesson 2

### Exercise 1

Tongue up on the Roof

Continue this exercise from Lesson 1. Hold your tongue tip on the spot for 5 minutes each time you practice.

### Exercise 2

Slurp and Swallow up on the Roof

The purpose of this exercise is to train your tongue to feel comfortable making contact on the roof of your mouth and still maintain a good swallow position. Bite your back teeth together, or keep them slightly apart, and smile real big, so your teeth show. Put your tongue tip on the spot. Now slurp your saliva and swallow. Do not let your tongue tip move from the spot. The slurping gives you some moisture to swallow and helps to suction your tongue up on the roof of your mouth. Get a glass of water and take sips in between slurps, as your mouth will get very dry. Do not slurp with water in your mouth. Hint: If you have difficulty with this exercise, you may be trying too hard. The slurp does not have to be a loud and long slurp. As soon as you slurp, your tongue is suctioned up on the roof of your mouth and you should swallow immediately. Do this exercise two times daily. Do it 5 times at each practice session.

### Exercise 3

Tongue taps

The purpose of this exercise is to stimulate the muscles in the blade of your tongue. Using your swizzle stick, chopstick, straw, spoon, pen or whatever item you chose to use in the last lesson, stick out your tongue and tap firmly on the top of the blade (middle section) of your tongue 60 times. Tap on the middle horizontal portion of the tongue—not the tip or the back—the middle portion. Practice it two times daily—that means 120 times daily.

You do NOT have to do Lesson 1 again. Each lesson prepares you for the next lesson. If, for some reason, you do not do the exercises two times daily for a particular lesson, do not proceed to the next lesson until you have completed the previous one. Do each lesson for at least 7 days.

### Lesson 3

### Exercise 1

Tense and Relax

This exercise will help you learn to control the muscles in the sides of your tongue. USE A MIRROR. Stick out your tongue and let it rest on your bottom lip. Tense the muscles so that your tongue is very narrow and pointed. The sides of your tongue are not curled up. Pretend that you are licking the chocolate icing off of the beater! Hold it there for 3 seconds. Now relax the muscles and hold it there for 3 seconds. Tense your tongue again for 3 seconds, then relax it for 3 seconds. Go back and forth. Do this 10 times at each practice session—two times daily.

### Exercise 2

Tongue Strokes and Taps

At each practice session, use a swizzle stick, toothbrush or straw to stroke each side of your tongue 10 times, as you did in Lesson 1. Also tap your tongue 60 times, as you did in Lesson 2. Do these 2 times daily

### Exercise 3

Activities up on the Roof×3

The purpose of this exercise is to help you get used to holding your tongue tip on the spot while you perform simple activities during the day. Each day, keep your tongue tip on the spot while you do 3 activities. These are activities that require concentration, making it more challenging for you to know where your tongue tip is resting. You will probably have to consciously put your tongue tip on the spot. Your teeth should be together and your lips should be closed. Your entire tongue should be flattened up against the roof of your mouth. This exercise is very important because you will purposefully plan to keep your tongue tip on the spot for 5 minutes—3 times per day. This will make you begin to be more aware of where your tongue tip rests when you are not talking or eating. Examples of activities: taking a shower, shaving, getting dressed, making the bed, washing dishes, cooking, any type of cleaning activity, working on the computer, etc. Driving, watching TV, and reading do not qualify for this exercise. You must be performing an activity, not just sitting. Of course, it will not hurt to do while just sitting! Make a sign to remind you to do your exercises.

### Exercise 4

Gargle

Gargling is a good exercise to help you relax and strengthen the muscles in the back of your tongue. Gargle every time you brush your teeth. You can gargle with mouthwash, plain water or with nothing in your mouth. You do not have to do Lesson 2 again if you practiced faithfully.

### Lesson 4

### Exercise 1

Tongue Strokes and Taps

At each practice session, use a swizzle stick or straw to stroke each side of your tongue 10 times, as you did in Lesson 1. Also tap your tongue 60 times, as you did in Lesson 2. Do these 2 times daily.

### Exercise 2

Activities up on the Roof×4

Last week you performed three activities each day while holding your tongue tip on the spot with your entire tongue resting against the roof of your mouth. This week, you will do FOUR activities each day. Each task should last about 5 minutes. Accordingly, it is preferable that you spend about 20 minutes a day with your tongue, lips, and teeth in the correct resting position. Make a sign to help you remember this exercise.

### Exercise 3

Gargle

Continue to gargle every time you brush your teeth.

### Exercise 4

Slow Tongue Pops

This exercise is very important in helping to strengthen the muscles in the blade (middle) of your tongue. Suction your entire tongue up on the roof of your mouth, like when you popped your tongue as a child. Look in the mirror and be sure that your lingual frenum (the muscle under your tongue) is stretched. Your mouth should be open but not real wide. Hold it there for 5 seconds and then pop it down slowly and forcefully. It doesn't have to be loud. Use the entire tongue, not just the tongue tip. Pop your tongue 20 times each time you practice. Practice two times daily.

### Exercise 5

Sleep Right

The purpose of this exercise is to train your tongue to stay on the spot while you sleep. This is very important. You will accomplish this by training your subconscious mind to keep your tongue in the correct position while sleeping. When you are ready to go to sleep (after you have watched TV or listened to the radio or read a book), put your tongue tip on the spot and try to get the entire tongue flattened up against the roof of your mouth as much as possible while still feeling comfortable. Try to create a vacuum between your tongue and the roof of your mouth. Tell yourself “tongue on the spot, tongue on the spot, tongue on the spot”. Close your eyes and go to sleep! You must do this exercise every night! Do not skip a night! This is the way your subconscious mind learns—it needs repetition, repetition, and repetition. Make a sign that says “Sleep Right” and put it by your bed to remind you.

You do not have to do Lesson 3 again if you practiced faithfully.

### Lesson 5

### Exercise 1

Rest Posture

Where your tongue “lives” in your mouth when you are not talking or eating is very important. Accordingly, the purpose of this exercise is to practice the correct resting posture of the tongue, lips and mandible. Raise your tongue tip to the spot. Close your teeth and lips. You may keep your teeth slightly apart if it is uncomfortable to keep them closed. Never clench your teeth. Your entire tongue should be flattened up against the roof of your mouth (or as much as possible to still remain comfortable). Maintain this correct resting posture while you watch one 30-minute TV program.

### Exercise 2

Trapped up on the Roof

This exercise will help your tongue become more used to staying flattened up against the roof of your mouth. Take a small sip of water. Put your tongue tip on the spot and raise your entire tongue against the roof of your mouth. Open your mouth and lean your head down to be sure that your tongue has formed a tight seal around the gum ridge. If no water drips out, you have done it correctly. Now, raise your head, bite your teeth together and swallow. Be sure your tongue tip does not slip off the spot when you swallow. Do this exercise about 10 times each time you practice. Practice two times daily. Hint: If you have difficulty with this exercise, you have probably taken a large sip. Try again with a smaller sip of water.

### Exercise 3

Tongue Drags on the Roof

Drag your tongue tip back along the hard and soft palate. Go as far as you can. Try it with your mouth open initially and then do it with your lips together. Do 10 tongue drags at each practice session for a total of 20 per day.

### Ecercise 4

Continue Sleep Right

Continue to tell yourself “tongue on the spot” every night before you go to sleep. This is very important. You do not have to do Lesson 4 again if you practiced faithfully.

### Lesson 6

### Exercise 1

Stretches up on the Roof

This exercise is similar to the slow tongue pops in Lesson 4. Suction your entire tongue up on the roof of your mouth and be sure that the lingual frenum (the muscle under your tongue) is stretched. Do NOT pop your tongue. While your tongue is up, open and close your jaw. Do this as many times as you can before your tongue falls down. Start with 10 stretches and increase your number until you can easily do 100 without stopping. Do this exercise two times daily.

### Exercise 2

Continue Tongue Drags on the Roof

Drag your tongue tip back along the hard and soft palate. Try it with your mouth open initially and then do it with your lips together. Do 10 tongue drags at each practice session for a total of 20 per day.

### Exercise 3

Where is your Tongue?

Your tongue should be on the spot and your teeth and lips should be closed at all times when you are not talking or eating. Your tongue should be flattened against the roof of your mouth as much as possible to remain comfortable. Start thinking about this!

### Exercise 4

Continue Sleep Right

Continue to tell yourself “tongue on the spot” every night before you go to sleep. This is very important.

You do not have to do Lesson 5 again if you practiced faithfully.

### Lesson 7

### Exercise 1

Tongue “Kicks”

The purpose of this exercise is to strengthen the back muscles of your tongue. When you say “kick”, the back of the tongue is humped up against your soft palate. Look in the mirror and watch the back of your tongue as you say, “Kick”. Raise and lower the back of your tongue 20 times at each practice session by saying “kick”, producing a hard “k” sound. Do this exercise two times daily.

### Exercise 2

Back Tongue Pressure

The purpose of this exercise is to stimulate the back of your tongue and make it easier to go up to the roof of your mouth. Place your fingertip on the back of your tongue, in the middle. If you go back too far, you may gag. Apply constant pressure with your index finger. Your tongue should resist this pressure. Hold for 5 seconds. Release. Do this exercise 20 times each time you practice. Be sure to practice 2 times daily.

### Exercise 3

Where is your Tongue?

Your tongue should be on the spot and your teeth and lips should be closed at all times when you are not talking or eating. Your tongue should be flattened against the roof of your mouth. Continue to think about this!

### Exercise 4

Continue Sleep Right

Continue to tell yourself “tongue on the spot” every night before you go to sleep. This is very important.

You do not have to do Lesson 6 again if you practiced faithfully.

### Maintenance

Your tongue should now be resting up against the roof of your mouth even when you are not thinking about it. It should now be a new habit. As time goes on, it is possible that you may slip back into your old tongue position and you do not want this to happen! This may happen if you have an allergy or sinus attack or if you get a cold. If, for some reason, you are unable to breathe through your nose for a while, you need to be sure that your tongue goes back on the spot as soon as your nose is clear.

You should continue to tell yourself to keep your tongue on the spot right before you go to sleep. Get into the habit of doing that.

For maintenance, you should consciously think about where your tongue is every morning on the way to work or at another specified time, during a specific activity. Make a sign to remind you to consciously think about your tongue position at least once per day. Pick a different exercise each day to practice. Watch a TV show with your tongue up on the roof of your mouth. You may not need to do a daily exercise if you know your tongue is always up on the roof of your mouth.

If your sleep pattern begins to change, take charge again and start the lessons over.

Treatment Results

In one study, a group of 23 patients were treated with an exercise plan as outlined in the application for a variety of symptoms including snoring and sleep apnea. The following results were obtained from the study.

The patients presented to the clinic with a number of symptoms related to sleep apnea.

The conditions of the patients included the following:
A-Have been diagnosed with sleep apnea
B-Breathe through mouth BEFORE
C-Breathe through mouth AFTER
D-Grind teeth BEFORE
E-Grind teeth AFTER
F-Have temporomandibular joint pain BEFORE
G-Have temporomandibular joint pain AFTER
H-Snore Loudly BEFORE
I-Snore Loudly AFTER
J-Wake up nonrefreshed and have trouble staying awake during the day BEFORE
K-Wake up nonrefreshed and have trouble staying awake during the day AFTER
L-Wake up during the night sometimes with the sensation of choking BEFORE
M-Wake up during the night sometimes with the sensation of choking AFTER
N-Wake up with headaches BEFORE
O-Wake up with headaches AFTER
P-Wake up sweating BEFORE
Q-Wake up sweating AFTER
R-Choke, gasp or hold breath during sleep BEFORE
S-Choke, gasp or hold breath during sleep AFTER
T-Have a dry mouth upon awakening BEFORE
U-Have a dry mouth upon awakening AFTER
V-Have difficulty concentrating BEFORE
W-Have difficulty concentrating AFTER
X-Have lapses in short-term memory BEFORE
Y-Have lapses in short-term memory AFTER
Z-Have heartburn BEFORE
AA-Have heartburn AFTER
AB-Have insomnia BEFORE
AC-Have insomnia AFTER
AD-Make frequent trips to bathroom during night BEFORE
AE-Make frequent trips to bathroom during night AFTER
AF-Have high blood pressure BEFORE
AG-Have high blood pressure AFTER
AH-Have Attention Deficit Disorder BEFORE
AI-Have Attention Deficit Disorder AFTER
AJ-Have restless leg syndrome BEFORE
AK-Have restless leg syndrome AFTER
AL-Have fibromyalgia BEFORE

AM-Have fibromyalgia AFTER

TABLE I
PATIENT SYMPTOMS BOTH BEFORE
AND AFTER TREAMENT

| Patient | A | B | C | D | E | F | G | H | I | J | K | L | M |
|---|---|---|---|---|---|---|---|---|---|---|---|---|---|
| 1 | | Yes | | Yes | | | | Yes | | Yes | | | |
| 2 | | Yes | | | | | | Yes | | | | | |
| 3 | | Yes | | | | | | Yes | | Yes | | | |
| 4 | | Yes | | | | | | Yes | | Yes | | | |
| 5 | | Yes | | Yes | | Yes | | Yes | | Yes | Yes | | |
| 6 | | Yes | | | | | | Yes | | Yes | | | |
| 7 | | | | Yes | | Yes | | Yes | | Yes | | | |
| 8 | | Yes | | | | | | Yes | Yes | Yes | | | |
| 9 | | | | | | Yes | | Yes | | Yes | | Yes | |
| 10 | | Yes | | Yes | | | | Yes | | Yes | | | |
| 11 | Yes | Yes | | | | | | Yes | | Yes | | Yes | |
| 12 | | | | Yes | | Yes | | Yes | | Yes | | Yes | |
| 13 | Yes | Yes | Yes | | | | | Yes | | Yes | | | |
| 14 | Yes | | | Yes | | Yes | | | | Yes | | | |
| 15 | | | | Yes | | | | | | Yes | | Yes | |
| 16 | | Yes | | | | | | | | | Yes | Yes | |
| 17 | | Yes | | | | | | Yes | | Yes | | | |
| 18 | | | | | | | | Yes | | | | | |
| 19 | Yes | Yes | | | | Yes | | Yes | | Yes | | | |
| 20 | | | | | | | | Yes | | Yes | | | |
| 21 | | Yes | | Yes | | | | Yes | | | | | |
| 22 | | Yes | | | | | | Yes | | | | | |
| 23 | | Yes | | Yes | | Yes | | Yes | | Yes | | | |
| Patient | N | O | P | Q | R | S | T | U | V | W | X | Y | Z |
| 1 | Yes | | | | Yes | | Yes | | | | | | |
| 2 | Yes | | | | | | | | | | | | |
| 3 | Yes | | | | | | Yes | | Yes | | Yes | | Yes |
| 4 | | | | | Yes | | Yes | | | | | | |
| 5 | | | | | | | | | | | | | |
| 6 | | | | | | | Yes | | | | Yes | | |
| 7 | | | | | | | | | Yes | | | | |
| 8 | | | | | Yes | | | | Yes | | Yes | | |
| 9 | Yes | | Yes | | Yes | | | | Yes | | Yes | | Yes |
| 10 | | | | | | | Yes | | | | | | |
| 11 | Yes | | | | Yes | | | | | | | | Yes |
| 12 | | | Yes | | | | Yes | | | | Yes | | |
| 13 | | | Yes | | Yes | | Yes | | Yes | | Yes | Yes | |
| 14 | | | | | | | | | Yes | | Yes | Yes | |
| 15 | | | Yes | | | | Yes | | | | | | Yes |
| 16 | | | Yes | Yes | | | Yes | Yes | | | | | |
| 17 | | | Yes | | Yes | | Yes | | | | | | Yes |
| 18 | | | | | Yes | | | | | | Yes | Yes | Yes |
| 19 | | | | | Yes | | Yes | | Yes | | Yes | | Yes |
| 20 | | | | | | | | | | | | | Yes |
| 21 | Yes | | Yes | | | | Yes | | | | Yes | | |
| 22 | | | | | | | Yes | | Yes | | | | |
| 23 | | | Yes | | | | Yes | | | | | | |
| Patient | AA | AB | AC | AD | AE | AF | AG | AH | AI | AJ | AK | AL | AM |
| 1 | | | | | | | | | | | | | |
| 2 | | | | Yes | | Yes | Yes | | | | | | |
| 3 | Yes | | | | | Yes | | | | | | | |
| 4 | | | | | | | | | | | | | |
| 5 | | | | Yes | Yes | | | | | | | | |
| 6 | | Yes | | Yes | Yes | | | | | Yes | | | |
| 7 | | Yes | | Yes | | | | Yes | Yes | | | | |
| 8 | | | | | | | | | | Yes | | | |
| 9 | | Yes | | Yes | | | | Yes | | | | | |
| 10 | | | | | | | | | | | | | |
| 11 | | | | | | Yes | | | | | | | |
| 12 | | | | | | | | | | | | | |
| 13 | | | | Yes | | Yes | Yes | | | | | | |
| 14 | | | | | | | | | | Yes | Yes | Yes | Yes |
| 15 | | Yes | | Yes | | | | | | | | | |
| 16 | | Yes | Yes | Yes | Yes | | | | | | | | |
| 17 | Yes | | | Yes | | | | | | | | | |
| 18 | Yes | | | | | | | | | | | | |
| 19 | | | | Yes | | | | | | | | | |
| 20 | | Yes | | Yes | | Yes | Yes | | | | | | |
| 21 | | | | | | | | | | | | | |
| 22 | | | | | | | | | | | | | |
| 23 | | | | | | | | | | | | | |

TABLE II
CHANGE IN SYMPTOMS

| | | # of | # of | | PERCENT |
|---|---|---|---|---|---|
| | | SYMPTOMS | SYMPTOMS | CHANGE IN | OF |
| | | BEFORE | AFTER | # of | TOTAL |
| Patient | MALE/FEMALE | TREATMENT | TREATMENT | SYMPTOMS | SUCCESS |
| 1 | F | 7 | 0 | 7 | 100% |
| 2 | M | 5 | 1 | 4 | 80% |
| 3 | M | 9 | 1 | 8 | 89% |
| 4 | M | 5 | 0 | 5 | 100% |
| 5 | F | 6 | 2 | 4 | 67% |
| 6 | M | 8 | 1 | 7 | 88% |
| 7 | F | 8 | 1 | 7 | 88% |
| 8 | M | 7 | 1 | 6 | 86% |
| 9 | F | 13 | 0 | 13 | 100% |
| 10 | F | 5 | 0 | 5 | 100% |
| 11 | F | 8 | 0 | 8 | 100% |
| 12 | F | 8 | 0 | 8 | 100% |
| 13 | M | 10 | 3 | 7 | 70% |
| 14 | F | 7 | 3 | 4 | 57% |
| 15 | F | 8 | 0 | 8 | 100% |
| 16 | F | 6 | 5 | 1 | 17% |
| 17 | F | 8 | 1 | 7 | 88% |
| 18 | M | 4 | 2 | 2 | 50% |
| 19 | F | 10 | 0 | 10 | 100% |
| 20 | F | 6 | 1 | 5 | 83% |
| 21 | F | 7 | 0 | 7 | 100% |
| 22 | F | 4 | 0 | 4 | 100% |
| 23 | F | 7 | 0 | 7 | 100% |

TABLE III

| | |
|---|---|
| AVERAGE SUCCESS | 85.26% |
| WITH TREATMENT | |
| Breathe through mouth | 93.75% |
| Grind Teeth | 100.00% |
| Tempormandibular pain | 100.00% |
| Snore Loudly | 95.00% |
| Wake nonrefreshed | 88.89% |
| Wake with sensation of choking | 100.00% |
| Headaches | 100.00% |
| Wake up sweating | 87.50% |
| Choke, gasp or hold breath | 100.00% |
| Dry mouth | 92.86% |
| Difficulty concentrating | 100.00% |
| Lapses in memory | 70.00% |
| Have heartburn | 62.50% |
| Have insomnia | 83.33% |
| Trips to the bathroom | 72.73% |
| High blood pressure | 40.00% |
| ADD | 50.00% |
| Restless leg syndrome | 66.67% |
| Fibromyalgia | 0.00% |

While the invention has been described in detail with reference to the preferred embodiments thereof, it will become apparent to one skilled in the art that various changes and modifications can be made and equivalents employed, without departing from the present invention.

1. A non-surgical method for treating sleep disorders such as snoring or sleep apnea which comprises:
instructing a patient having a sleep disorder to perform tongue exercises directed to positioning the tongue in a predetermined location on the hard palate behind the upper front teeth.

2. The method of claim 1, further comprising instructing the patient to perform additional exercises directed toward strengthening the muscles of the tongue.

3. The method of claim 1, further comprising instructing the patient to perform additional exercises directed toward stimulating the muscles of the tongue.

4. The method of claim 1, further comprising instructing the patient to perform additional exercises directed toward controlling the muscles of the tongue.

5. The method of claim 1, further comprising instructing the patient to perform additional exercises directed toward teaching the tip of the tongue to rest in the predetermined location during everyday activities.

6. The method of claim 1, wherein one of the exercises directed to positioning the tongue in a predetermined location comprises:
placing the tip of the patient's tongue against the patient's hard palate right behind the upper front teeth; and
holding the tip of the tongue in that position for at least about 60 seconds.

7. The method of claim 2, wherein one of the exercises directed toward strengthening muscles of the tongue comprises:
placing the tip of the tongue against the hard palate;
sucking in slightly; and
repeating the sucking motion for at least about 30 seconds.

8. The method of claim 3, wherein one of the exercises directed toward strengthening muscles of the tongue comprises:
sticking a patients tongue out of the mouth and relaxing the tongue against a patient's lower lip;
placing an elongated object against a side of the tongue beginning in a back portion of the patient's mouth; and
pulling the object forward to stroke the side of the tongue.

9. A non-surgical method for treating sleep disorders such as snoring or sleep apnea symptoms which comprises:
instructing a patient having a sleep disorder to perform tongue exercises directed to positioning the tongue in a predetermined location on the hard palate and directly behind the upper front teeth; and
instructing the patient to perform at least one additional tongue exercise selected from the group consisting of:
a. an exercise for strengthening the muscles of the tongue;
b. an exercise for stimulating the muscles of the tongue;
c. an exercise for controlling the muscles of the tongue;
d. an exercise for teaching the muscles of the tongue to rest in the predetermined location during everyday activities.

10. A non-surgical method of treating sleep disorders such as snoring or sleep apnea which comprises;
instructing a patient having a sleep disorder to perform tongue exercises that train the patient's tongue to rest against the hard palate directly behind the upper front teeth, wherein the patient performs at least one of the following exercises at least once a day for at least two days:
a. tongue up on the roof;
b. tongue tip exercises;
c. tongue strokes.

11. The method of claim 10, further comprising instructing the patient to perform at least one of the following additional tongue exercises at least once a day for at least two days:
a. tongue up on the roof;
b. slurp and swallow up on the roof;
c. tongue taps.

12. The method of claim 10, further comprising instructing the patient to perform at least one of the following additional tongue exercises at least once a day for at least two days:
a. tense and relax;
b. tongue strokes and taps;
c. activities up on the roof×3;
d. gargle.

13. The method of claim 10, further comprising instructing the patient to perform a maintenance program after the patient performs the tongue exercises, wherein the maintenance program is to rest the patient's tongue on the hard palate directly behind the upper front teeth when the patient is not talking, eating or drinking.

14. The method of claim 10, wherein the tongue up on the roof exercise comprises the following steps:
placing the tip of the patient's tongue against the hard palate directly behind the upper front teeth; and
holding the tip of the tongue in that position for at least about 60 seconds.

15. The method of claim 10, wherein the tongue tip exercise comprises the following steps:
placing the tip of the tongue against the hard palate;
sucking in slightly; and
repeating the sucking motion for at least about 30 seconds.

16. The method of claim 10, wherein the tongue strokes exercise comprises the following steps:
sticking a patients tongue out of the mouth and relaxing the tongue against the patient's lower lip;
placing an elongated object against a side of the tongue and beginning in the very back and
pulling the object forward to stroke the side of the tongue.

17. A method for treating sleep disorders by training a person's tongue to rest against a roof of the person's mouth, the method comprising the person performing at least one of the following exercises at least once a day for at least two days:
an exercise for teaching the tongue to rest against the roof of the mouth;
an exercise for strengthening the muscles of the tongue;
an exercise for stimulating the muscles of the tongue;
an exercise for controlling the muscles of the tongue; and
an exercise for training the tip of the tongue to rest against the roof of the mouth during everyday activities.

18. The method of claim 17, further comprising the person performing an exercise for training the tip of the tongue to rest against the roof of the mouth while sleeping.

19. The method of claim 17, further comprising the person performing a plurality of the exercises for at least seven days.

20. A non-surgical method for treating sleep disorders such as snoring or sleep apnea, which comprises:
performing a first series of tongue exercises to train a patient's tongue to rest on the hard palate directly behind the upper front teeth; and
repeating the first series of tongue exercises daily for at least two days.

21. The method of claim 20, wherein the first series of exercises includes at least one exercise selected from the following group of exercises: Tongue up on the Roof, Tongue Tip Exercise, Tongue Strokes, Slurp and Swallow up on the Roof, Tongue Taps, Tense and Relax, Activities up on the Roof×3 (or ×4), Gargle, Slow Tongue Pops, Sleep Right, Rest Posture, Trapped up on the Roof, Tongue Drags on the Roof, Stretches up on the Roof, Where is your Tongue?, Tongue Kicks, and Back Tongue Pressure.

22. The method of claim 20, further comprising performing a second series of tongue exercises.

23. The method of claim 22, further comprising repeating the second series of tongue exercises for at least two days.

24. The method of claim 22, wherein the second series of exercises includes at least one exercise selected from the following group of exercises: Tongue up on the Roof, Tongue Tip Exercise, Tongue Strokes, Slurp and Swallow up on the Roof, Tongue Taps, Tense and Relax, Activities up on the Roof×3 (or ×4), Gargle, Slow Tongue Pops, Sleep Right, Rest Posture, Trapped up on the Roof, Tongue Drags on the Roof, Stretches up on the Roof, Where is your Tongue?, Tongue Kicks, and Back Tongue Pressure.

25. The method of claim 23, further comprising performing a third series of tongue exercises.

26. The method of claim 25, further comprising repeating the third series of tongue exercises for at least two days.

27. The method of claim 20, further comprising performing a maintenance program.

28. The method of claim 27, wherein the maintenance program includes at least one exercise selected from the following group: Tongue up on the Roof, Tongue Tip Exercise, Tongue Strokes, Slurp and Swallow up on the Roof, Tongue Taps, Tense and Relax, Activities up on the Roof×3 (or ×4), Gargle, Slow Tongue Pops, Sleep Right, Rest Posture, Trapped up on the Roof, Tongue Drags on the Roof, Stretches up on the Roof, Where is your Tongue?, Tongue Kicks, and Back Tongue Pressure.

29. A non-surgical method for treating sleep disorders such as snoring or sleep apnea, the method comprising the steps of:
performing at least one exercise for training the tongue to rest against a roof of a mouth, wherein the at least one exercise is performed at least once a day, wherein at least one exercise includes one of the following:
Tongue up on the Roof, Tongue Tip Exercise, Tongue Strokes, Slurp and Swallow up on the Roof, Tongue Taps, Tense and Relax, Activities up on the Roof×3 (or ×4), Gargle, Slow Tongue Pops, Sleep Right, Rest Posture, Trapped up on the Roof, Tongue Drags on the Roof, Stretches up on the Roof, Where is your Tongue?, Tongue Kicks, and Back Tongue Pressure.

30. The method of claim 29, further comprising performing at least two exercises for a period of not less than 2 days.

31. The method of claim 29, further comprising performing at least two exercises for a period of time, wherein the at least two exercises are different from the at least two exercises previously performed.

32. A non-surgical method for treating sleep disorders comprising:
instructing a patient having a sleep disorder to perform an exercise directed to positioning the tongue in a predetermined location on the hard palate behind the upper front teeth by performing an exercise comprising:
placing the tip of the patient's tongue against the patient's hard palate right behind the upper front teeth; and
holding the tip of the tongue in that position for at least about 30 seconds.

33. A method of claim 32, further comprising instructing the patient to perform at least one of the following exercises:
a. Sleep Right; and
b. Activities up on the Roof×3.

34. A method of claim 32, further comprising instructing the patient to perform at least one of the following exercises:
a. Sleep Right;
b. Activities up on the Roof×3;
c. Tongue Tip Exercises; and
d. Stretches up on the Roof.

35. A medium comprising instructions for performing a non-surgical method for treating sleep disorders such as snoring or sleep apnea which comprises:
instructing a patient having a sleep disorder to perform tongue exercises directed to positioning the tongue in a predetermined location on the hard palate behind the upper front teeth.
